(19) United States
(12) Patent Application Publication
(10) Pub. No.: US 2025/0354126 A1
(43) Pub. Date: Nov. 20, 2025

(54) HYDROGEL-BASED CELL ENCAPSULATION METHOD, A CELL OR CELL-ENCAPSULATING POLYMERIZED MICROGEL AND A SYSTEM THEREOF, AND KIT THEREOF

(71) Applicant: City University of Hong Kong, Hong Kong (CN)

(72) Inventors: Mengsu YANG, Hong Kong (CN); Xiaoyu ZHOU, Hong Kong (CN); Yuan WANG, Hong Kong (CN); Xuhua HOU, Hong Kong (CN)

(21) Appl. No.: 18/869,329

(22) PCT Filed: May 23, 2023
(86) PCT No.: PCT/CN2023/095763

§ 371 Date: Nov. 26, 2024

(30) Foreign Application Priority Data
May 30, 2022 (CN) 202210600576.6

(51) Int. Cl. C12N 5/09

(52) U.S. Cl. CPC C12N 5/0693; C12N 2533/74; C12N 2537/10

(57) ABSTRACT

Hydrogel-based cell encapsulation method, a cell-encapsulating polymerized microgel prepared therefrom, and a kit thereof. The hydrogel-based cell encapsulation method can include: (a) providing a cell suspension as an aqueous phase comprising cells, alginate and a culture medium; (b) preparing an emulsion using the cell suspension and an oil phase through a droplet microfluidic device such that one or more of the cells are each encapsulated in at least a portion of the droplets thereby forming an emulsion comprising cell-encapsulating droplets and empty droplets; (c) incubating the emulsion, and then cross-linking alginate in the cell-encapsulating droplets in the presence of a cross-linker to form cell-encapsulating polymerized microgels; and (d) optionally, demulsifying, separating and collecting the cell-encapsulating polymerized microgels.

### CROSS-REFERENCE TO RELATED APPLICATIONS

This application claims priority from China Patent Application Number 202210600576.6, filed on May 30, 2022, the content of which is hereby incorporated by reference in its entirety.

### TECHNICAL FIELD

The present disclosure relates to a hydrogel-based cell encapsulation method, a cell or cell-encapsulating polymerized microgel, and kit thereof.

### BACKGROUND

In recent years, droplet microfluidic technology has been widely used in cell analysis and research due to its advantages of miniaturization, low cost, high sensitivity, high specificity, high throughput, etc. and has become a powerful tool, especially for single cell manipulation and analysis. In droplet microfluidic devices, information at the level of cell genomics, transcriptomics, proteomics, or metabolomics can be obtained easily by encapsulating cells into picoliter-to-nanoliter-sized droplets.

Hydrogel materials have been introduced into droplet microfluidic devices for cell research due to their unique solution-gel transition properties. The hydrogel materials can be contained in the aqueous phase of the emulsion as polymerization precursors, and solution-gel transition is performed after droplets are generated using droplet microfluidic devices, so that uniform hydrogel particles for encapsulating cells or molecules can be obtained in a high throughput way.

During the cell encapsulation process, the encapsulation efficiency is limited by the random process determined by the Poisson distribution. To obtain a higher cell/single-cell encapsulation rate, it is necessary to dilute the suspension to reduce the cell density. The side effect is that a significant portion of empty droplets are generated during the droplet encapsulation process, which reduces the efficiency of droplet-based cell encapsulation.

Currently, there are generally two methods to obtain deterministic cell encapsulation using droplet microfluidic devices: The first is to perform secondary droplet sorting after encapsulation to obtain the pure population of droplets with cells; the second is to control the arrangement of cells in the aqueous channel before encapsulation, so that orderly encapsulation is carried out by matching the rate at which cells arrive at the droplet generation structure to the frequency of droplet generation, while attempting to make every droplet generated contain cells/single cells.

No matter which of the methods described above is adopted, it is necessary to screen or manipulate droplets by one of them, i.e., based on the difference in droplet size/laminar flow properties or with the need for an external force field (e.g., magnetic force, dielectrophoretic force, electrodynamic force, etc.) assistance, so as to obtain a higher cell encapsulation rate. Among them, the external force field-assisted means is more conducive to obtaining a high cell encapsulation rate. However, with the help of an external force field, it is not only necessary to set up more complex experimental devices, such as fluorescence detection module, surface acoustic wave module, etc., but it is also unavoidably necessary to label the cells and introduce an external force field to manipulate them, which will expose the cells to external stresses, thereby deteriorating cell growth and living conditions, thus potentially changing cell behavior, and leading to errors in subsequent cell analysis. Therefore, there is an urgent need for a simpler, more efficient and more accurate deterministic cell encapsulation method to improve the process of analysis and research at single cell level.

### SUMMARY

The present disclosure provides a hydrogel-based cell encapsulation method without the need to label the cells and/or introduce an external force field to manipulate the cells, thereby eliminating the need to use complex experimental devices and avoiding the possibility of cell behavior being affected by the source. Moreover, the method of the present disclosure also enables a high cell encapsulation rate, thereby providing a simpler, more efficient, and more accurate deterministic cell encapsulation method.

In a first aspect, provided herein is a hydrogel-based cell encapsulation method comprising:
(a) providing a cell suspension as an aqueous phase comprising cells, alginate and a culture medium;
(b) preparing an emulsion using the cell suspension and an oil phase through a droplet microfluidic device such that one or more of the cells are each encapsulated in at least a portion of the droplets thereby forming an emulsion comprising cell-encapsulating droplets and empty droplets;
(c) incubating the emulsion, and then cross-linking alginate in the cell-encapsulating droplets in the presence of a cross-linker to form cell-encapsulating polymerized microgels; and
(d) optionally, demulsifying, separating and collecting the cell-encapsulating polymerized microgels.

In certain embodiments, in step (a), the cell suspension further comprises a water-soluble divalent metal salt selected from the group consisting of calcium chloride, calcium bromide, calcium iodide, calcium nitrate, calcium chlorate, calcium perchlorate, calcium bicarbonate, calcium dihydrogen phosphate, calcium acetate, calcium gluconate, calcium hydrogen phosphate, calcium lactate, calcium nitrate, barium chloride, barium sulfate, barium nitrate, barium carbonate, barium cyanide, and a combination thereof.

In certain embodiments, the water-soluble divalent metal salt is present in the cell suspension at a concentration sufficient to crosslink the alginate present in the cell-encapsulating droplets when the pH of the cell-encapsulating droplets is reduced during incubation of the emulsion.

In certain embodiments, the concentration of the water-soluble divalent metal salt in the cell suspension is 2-8 mM, 3.5-7 mM, or 6-7 mM.

In certain embodiments, the step (c) comprises: subjecting the emulsion to a first incubation for 0.5-24 hours or 1.5-3 hours; and subjecting the emulsion to a second incubation for 10-60 minutes or 15-35 minutes in the presence of a cross-linker, to crosslink the alginate in the cell-encapsulating droplets.

In certain embodiments, the cross-linker is added to the emulsion before the first incubation or to the emulsion after the first incubation and before the second incubation.

In certain embodiments, the cross-linker is added to the oil phase of the emulsion or to the aqueous phase of the emulsion.

In certain embodiments, in step (c), when the first incubation is completed, the pH of the cell-encapsulating droplet is less than or equal to 6.5 or has a pH between 6-6.5.

In certain embodiments, the cross-linker is calcium sulfate, barium sulfate, or a combination thereof.

In certain embodiments, the cross-linker is in the form of a powder, a crystal, or a nanoparticle.

In certain embodiments, each of the cell-encapsulating polymerized microgels obtained in step (d) comprise: a single cell, two cells, or more than two cells; and separating and collecting the cell-encapsulating polymerized microgels comprises collecting and separating the cell-encapsulating polymerized microgels comprising two cells and two or more cells first, and then collecting and separating the cell-encapsulating polymerized microgels encapsulating single cells.

In certain embodiments, the cells comprise human cells, mammalian cells, cancer cells, somatic cells, spleen cells, stem cells, or germ cells.

In certain embodiments, in step (a), the concentration of the cells in the cell suspension is 5×10⁵ to 3×10⁶ cells/ml.

In certain embodiments, in step (b), the oil phase is selected from the group consisting of a fluorinated oil, a silicone oil, a rapeseed oil, a mineral oil, a droplet oil, or any combination thereof.

In certain embodiments, the emulsion comprises cell-encapsulating polymerized microgels and empty hydrogel particles at a ratio between 75:25 to 90:10, respectively.

In certain embodiments, the 70-80% of the cell-encapsulating polymerized microgels comprise a single cell.

In certain embodiments, separating and collecting the cell-encapsulating polymerized microgels comprises separating the cell-encapsulating polymerized microgel based on their relative density.

In certain embodiments, separating and collecting the cell-encapsulating polymerized microgels comprises centrifugation, sieving, or surface adhesion.

In a second aspect, provided herein is a cell-encapsulating polymerized microgel prepared by the cell encapsulation method described herein.

In a third aspect, provided herein is a kit for conducting the cell encapsulation method described herein, the kit comprising: alginate; a culture medium; an oil phase; a cross-linker; and optionally instructions for use.

The present disclosure also provides a cell-encapsulating polymerized microgel and a kit. Since the cells are not labeled and/or introduced to an external force field to manipulate them during the preparation process, the cells are maximized to maintain their intrinsic behavior patterns and the intrinsic levels of various substances therein.

The present disclosure also provides the use of the cell or cell-encapsulating polymerized microgel as described above in the preparation of a formulation for use in cell research, such as cell life/death sorting, cell metabolism analysis, and stem cell sorting, and liquid biopsy analysis.

The present disclosure also provides a kit for use, for example, in the hydrogel-based cell encapsulation method described in the present invention.

### BRIEF DESCRIPTION OF DRAWINGS

The above and other objects and features of the present disclosure will become apparent from the following description of the disclosure, when taken in conjunction with the accompanying drawings.

FIG. 1 shows upper panel (A) shows a schematic flow chart of the deterministic cell encapsulation method provided by certain embodiments described herein; lower panel (B) shows the trends of fluorescence intensity and fluorescence image of WBCs-containing droplets (top) and A549-containing droplets (bottom).

FIG. 2 shows the results for the number, diameter, and circularity of the cell-encapsulating polymerized microgels under different concentrations of CaCl₂ in the in the cell suspension.

FIG. 3 shows the results for the number, diameter, and circularity of the gel particles as a function of the first incubation time.

FIG. 4 shows the effect of the cross-linker calcium sulfate and its different morphologies on the number, diameter and circularity of the gel particles.

FIG. 5 shows the results for the number, diameter, and circularity of the gel particles as a function of the second incubation time.

FIG. 6 show the results of cell encapsulation efficiency in droplets after encapsulation and cell-encapsulating polymerized microgels after sorting to remove empty droplets not containing cell to obtain the lung cancer A549 cell line at different cell concentrations: (A) 3×10⁶ cells/mL; (B) 1×10⁶ cells/mL; and (C) 5×10⁵ cells/mL, wherein the broken line with open circles corresponds to the left ordinate, indicating the percentage of cell encapsulation rate in the droplet, while the broken line with solid circles corresponds to the right ordinate, indicating the percentage of cell encapsulation rate in the cell-encapsulating polymerized microgels.

FIG. 7 show the results of encapsulation rates in droplets and hydrogel particles obtained for the white blood cells (WBCs) at different cell concentrations: (A) 1×10⁶ cells/mL; and (B) 5×10⁵ cells/mL, wherein the broken line with open circles corresponds to the left ordinate, indicating the percentage of cell encapsulation rate in the droplet, while the broken line with solid circles corresponds to the right ordinate, indicating the percentage of cell encapsulation rate in the cell-encapsulating polymerized microgels (C) shows the encapsulation rate for a cell mixture dissected from spleen tissue of mice.

FIG. 8 shows the (A) cell viability and (B) cell proliferation ability after release.

FIG. 9 shows the components of two exemplary commercial oil phase comprising a surfactant: 008-FluoroSurfactant in HFE-7500 and 008-FluoroSurfactant in FC40.

### DETAILED DESCRIPTION

The present disclosure relates to a cell encapsulation method, which does not require labeling/external force field assistance, for deterministic encapsulation of cells in hydrogel particles. The present disclosure breaks down the complicated process of cell/single-cell encapsulation into two basic elements: one is to use the inherent metabolism activities of cells to identify the droplets containing cells without labeling, and the other is to use the difference in the physical properties of the hydrogel particles and the uncrosslinked empty droplets to sort cell-encapsulating polymerized microgels from particles without encapsulated cells without external force fields, and enabling the batch collection of cell-encapsulating polymerized microgels containing different numbers or types of cells as desired.

Throughout the present disclosure, unless the context requires otherwise, the word “comprise” or variations such as “comprises” or “comprising”, will be understood to imply the inclusion of a stated integer or group of integers but not the exclusion of any other integer or group of integers. It is also noted that in this disclosure and particularly in the claims and/or paragraphs, terms such as “comprises”, “comprised”, “comprising” and the like can have the meaning attributed to it in U.S. Patent law; e.g., they can mean “includes”, “included”, “including”, and the like; and that terms such as “consisting essentially of” and “consists essentially of” have the meaning ascribed to them in U.S. Patent law, e.g., they allow for elements not explicitly recited, but exclude elements that are found in the prior art or that affect a basic or novel characteristic of the present invention.

Furthermore, throughout the present disclosure and claims, unless the context requires otherwise, the word “include” or variations such as “includes” or “including”, will be understood to imply the inclusion of a stated integer or group of integers, but not the exclusion of any other integer or group of integers.

The use of the singular herein includes the plural (and vice versa) unless specifically stated otherwise. In addition, where the use of the term “about” is before a quantitative value, the present teachings also include the specific quantitative value itself, unless specifically stated otherwise.

As used herein, the term “about” refers to a ±10%, ±7%, ±5%, ±3%, ±1%, or ±0% variation from the nominal value unless otherwise indicated or inferred.

As used herein “droplet” refers an isolated aqueous phase within an oil phase having any shape, for example cylindrical, spherical, ellipsoidal, irregular shapes, etc. Generally, in emulsions described herein, aqueous droplets are spherical or substantially spherical in an oil phase, continuous phase.

In one aspect, provided herein is a hydrogel-based cell encapsulation method comprising:
(a) preparing a cell suspension as an aqueous phase comprising cells, alginate and a culture medium;
(b) preparing an emulsion using the cell suspension and an oil phase through a droplet microfluidic device such that at least part of the droplets of the emulsion are encapsulated with cells; and
(c) incubating the emulsion, and then cross-linking alginate in the cell-encapsulating droplets in the presence of a cross-linker to form cell-encapsulating polymerized microgels.

In certain embodiments, the method may also optionally include (d) demulsifying, separating and collecting the cell-encapsulating polymerized microgels.

In steps (a) and (b), the method utilizes the droplet microfluidic device to generate the emulsion together with the oil phase, with the cell suspension comprising alginate and culture medium as the aqueous phase, so that the emulsion has droplets of the aqueous phase that at least partially encapsulate cells. Methods for using droplet microfluidic devices to make emulsions and dispersed droplets is one well known to those skilled in the art.

In certain embodiments of the method, the alginate in step (a) can be sodium alginate, potassium alginate, or a complex of alginic acid and other materials (e.g., a complex of alginic acid and collagen). In certain embodiments, the alginate in step (a) is sodium alginate.

In certain embodiments of the method, the concentration of the cell suspension in step (a) is 5×10⁵ to 3×10⁶ cells/ml or 1×10⁶ to 3×10⁶ cells/ml. In certain embodiments of the method, in step (a), the cell suspension further comprises a water-soluble divalent metal salt, such as one or more of barium chloride, calcium chloride, calcium bromide, calcium iodide, calcium nitrate, calcium chlorate, calcium perchlorate, calcium bicarbonate, calcium dihydrogen phosphate, calcium acetate, calcium gluconate, calcium hydrogen phosphate, calcium lactate, calcium nitrate, barium chloride, barium sulfate, barium nitrate, barium carbonate, barium cyanide, and the like. Without wishing to be bound by theory, it is believed that the water-soluble divalent metal salt partially pre-cross-links alginate to maintain the properties of the droplets and shorten the time for the first incubation of the droplets, so as to ensure that the hydrogel has enough cross-linkers to achieve complete polymerization. The concentration of the water-soluble divalent metal salt may be 2-8 mM or any concentration value or range therebetween. In certain embodiments, the concentration of the water-soluble divalent metal salt is 3.5-7 mM, 6-7 mM or 6.54 mM.

In certain embodiments, the water-soluble divalent metal salt has higher water solubility than the cross-linker.

Cells useful in the methods described herein are not particularly limited. As such, the cell can be any type of cell. In certain embodiments of the method described herein, the cells comprise human cells or mammalian cells, such as cancer cells, somatic cells, spleen cells, stem cells, or germ cells (eggs or sperm).

According to the method described herein, the oil phase that can be used in step (b) can be an oil phase that is substantially immiscible with the cell suspension and can generate stable water-in-oil droplets. In certain embodiments, the oil phase is biocompatible. In certain embodiments, the oil phase is an oil, a non-polar solvent, a fluorinated oil, a silicone oil, a rapeseed oil, a mineral oil, a fluorinated surfactant, a fluorocarbon, a silicone oil, decane, tetradecane, hexadecane, a commercial droplet oil (also known as a droplet generation oil), such as Biorad droplet-forming oil, and the like, or any combination thereof. Suitable oil phases are known to those skilled in the art in which the aqueous phase spontaneously leads to the formation of water droplets or isolated volumes or compartments surrounded by the oil phase.

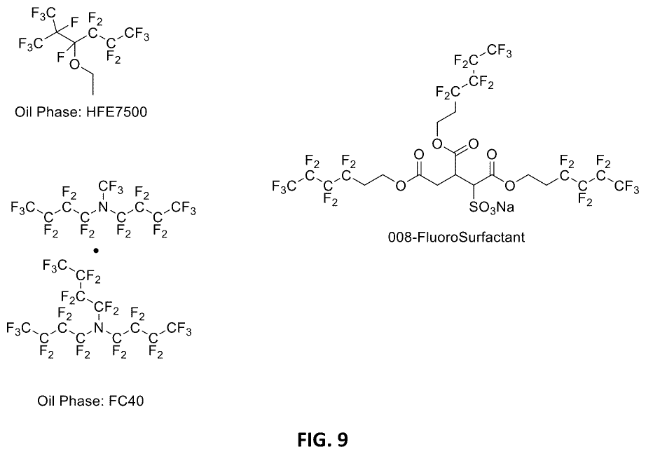

In certain embodiments, the oil phase further comprises one or more surfactants. The surfactant can be sorbitan-based carboxylic acid esters, such as sorbitan monolaurate (Span 20), sorbitan monopalmitate (Span 40), sorbitan monostearate (Span 60), sorbitan monooleate (Span 80), Tween 20 (polysorbate 20), and Tween 40 (polysorbate 40), polyoxyethylenated alkylphenols, such as Triton X-100, polyoxyethylenated straight chain alcohols, polyoxyethylenated polyoxypropylene glycols, polyoxyethylenated mercaptans, long chain carboxylic acid esters, Triton X-100, etc., or a fluorine-containing surfactant, such as 008-FluoroSurfactant (FIG. 9).

Exemplary commercial oil phases comprising surfactants include, but are not limited to, QX200™ Droplet Generation Oil sold by Bio-Rad™, FluoroSurf™ surfactant in fluorinated oil sold under the tradename HFE-7500 by RAN Biotechnologies™, Pico-Surf™ 1 sold by Dolomite Microfluidics™, and the like.

It has been surprisingly discovered that the accumulation of cellular metabolites, e.g., respiration-generated CO₂ or lactic acid, can cause a decrease in the pH of the extracellular environment, and thus, for a cell-encapsulating droplet (aqueous phase), as the cellular metabolites accumulate, the pH of the droplet will decrease accordingly. Therefore, according to the methods described herein, during the first incubation of step (c), the pH of the cell-encapsulating droplets will decrease with the accumulation of the cellular metabolites, however, the pH of the droplets without cells will not change. Thus, the present method exploits the metabolic properties of cells themselves as a means of identifying droplets encapsulating cells without labeling and/or manipulation by external force fields.

The amount of time required to incubate the emulsion can thus depend on a number of factors, such as the type of cells present in the emulsion and concentration, the cross-linker used and its concentration, the concentration and average molecular weight of alginate, and the like. The selection of the appropriate incubation time and the number of incubations required is well within the skill or a person of ordinary skill in the art. Accordingly, step (c) can comprise subjecting the emulsion to one or more incubations for a period of time necessary for the pH of the cell-encapsulating droplets to drop to a sufficient pH to solubilize a adequate quantity of the cross-linker to selectively crosslink the alginate in the cell-encapsulating droplets.

In certain embodiments of the method, the step (c) comprises: subjecting the emulsion to a first incubation for 0.5-24 hours, 1-3.5 hours, or 1.5-3 hours; subjecting the emulsion to a second incubation for 10-60 minutes, 20-40 minutes, or 15-35 minutes in the presence of a cross-linker, to selectively crosslink the alginate in the cell-encapsulating droplets.

In certain embodiments of the method, the time of the first incubation in step (c) is 1.5-3 hours, or any period of time therebetween, for example 1.5, 1.6, 1.7, 1.8, 1.9, 2, 2.1, 2.2, 2.3, 2.4, 2.5, 2.6, 2.7, 2.8, or 2.9 hours. The incubation condition can be adjusted accordingly the types of cell encapsulated, for example, 37° C. and 5% CO₂ for cancer cells.

The necessary pH required to achieve sufficient solubility of the cross-linker in the droplet to crosslink the alginate can depend on the type of cross-linker used, the concentration of the cross-linker, and the concentration of the alginate present in the droplets. The selection of the necessary pH required to initiate the crosslinking of the alginate present in the droplets is well within the skill of a person of ordinary skill in the art. Thus, in certain embodiments, step (c) can comprise incubating the emulsion one or more times for a sufficient period of time for the pH of the cell-encapsulating droplets to decrease in pH thereby increasing the solubility of the cross-linker in the cell-encapsulating droplets and selectively crosslinking the cell-encapsulating droplets.

In certain embodiments of the method, in step (c), when the first incubation is completed, the pH of the cell-encapsulating droplet is less than or equal to less than or equal to 7, less than or equal to 6.9 less than or equal to 6.8, less than or equal to 6.7, less than or equal to 6.6, less than or equal to 6.5, less than or equal to 6.4, less than or equal to 6.3, less than or equal to 6.2, less than or equal to 6.1, less than or equal to 6. In certain embodiments of the method, in step (c), when the first incubation is completed, the pH of the cell-encapsulating droplet is 6-7, 6-6.9, 6-6.8, 6-6.7, 6-6.6, 6-6.5, 6-6.4, 6-6.3, 6-6.2, 6-6.1, 6.1-6.9, 6.2-6.8, 6.3-6.7, or 6.4-6.6. In certain embodiments, of the method, in step (c), when the first incubation is completed, the pH of the cell-encapsulating droplet is about 6.5.

Next, in step (c), once the pH of the cell-encapsulating droplet drops to a sufficient pH, the alginate contained inside the droplets encapsulating the cells is subjected is crosslinked. For this purpose, a cross-linker may be added to the emulsion described herein, for example to the aqueous or oil phase of the emulsion. In certain embodiments of the method, a cross-linker may be added in the oil phase to allow a cross-linking reaction of the alginate to occur. More specifically, the cross-linker may be mixed into the same or different oil phase substance as the oil phase substance in the emulsion to obtain a mixture, and the mixture may be added to the above emulsion. For example, the cross-linker can be mixed into the oil phase at a concentration of more than 20 mM to obtain a mixture.

In certain embodiments of the method, the cross-linker is added to the emulsion before the first incubation or added to the emulsion after the first incubation and before the second incubation.

In certain embodiments of the method, the cross-linker is a divalent metal ion sulfate, such as CaSO₄, MgSO₄, SrSO₄, BaSO₄, ZnSO₄, or the like, or mixtures thereof. The solubility of the divalent metal ion sulfate increases with the decrease of pH, and it has also been found that its solubility is sufficient to cause the alginate to undergo a cross-linking reaction at a reduced pH (e.g., less than or equal to 6.5 or 6-6.5) to obtain a gel.

In embodiments where the cross-linker is added to the emulsion before the first incubation, preferably the cross-linker does not cross-link the alginate before the first incubation (i.e., when the extracellular environment has not changed) or during the first incubation (i.e., when the pH of the extracellular environment has not dropped low enough) so as to realize the objective of using the metabolic properties of the cells themselves to differentiate aqueous-phase droplets encapsulating cells.

As an example of the cross-linker used in the method, calcium sulfate, barium sulfate, or a combination thereof may be enumerated. In certain embodiments, the divalent metal ion sulfate may be in the form of a powder, a crystal, or any nanostructure (a nanotube, a nanoparticle, etc.). Suitable cross-linkers, e.g., those described above, have good biocompatibility and have solubility suitable for use in the method described herein as well as the degree of change of solubility with pH.

In certain embodiments, the divalent metal ion sulfate is in the form of nanoparticles. The nanoparticles can have a particle size of 100-999 nm or 200-500 nm. In certain embodiments, the nanoparticles are prepared by the divalent metal ion sulfate and the oil phase used in step (b).

In certain embodiments of the method, the concentration of the cross-linker in the emulsion has a concentration of 0.1-1 g/ml, 0.1-0.9 g/ml, 0.1-0.8 g/ml, 0.1-0.7 g/ml, 0.1-0.6 g/ml, 0.1-0.5 g/ml, 0.1-0.4 g/ml, 0.1-0.4 g/ml, 0.2-0.3 g/ml, 0.2-0.29 g/ml, 0.2-0.28 g/ml, 0.2-0.27 g/ml, 0.2-0.26 g/ml, 0.21-0.26 g/ml, 0.21-0.25 g/ml, 0.22-0.25 g/ml, or 0.22-0.24 g/ml. In certain embodiments of the method, the concentration of the cross-linker in the emulsion has a concentration of about 0.23 g/ml, added in the oil phase, and the cross-linker is calcium sulfate nanoparticles.

According to the method described herein, the pH in the cell-encapsulating droplets can decrease after the first incubation due to the metabolism of the cells themselves, resulting in an increase in the solubility of the cross-linker in the aqueous phase sufficient to initiate cross-linking of the alginate during the second incubation, such that the cell-encapsulating aqueous-phase droplets form gel particles containing cells, while the properties of the non-cell-encapsulating aqueous droplets remains unchanged.

In step (c), the second incubation is performed for 20-40 minutes, 15-35 minutes, or 16, 17, 18, 19, 20, 21, 22, 23, 24, 25, 26, 27, 28, 29, 30, 31, 32, 33, 34, 35 minutes, or any range or value therebetween.

Advantageously, the methods described herein can selectively crosslink cell-encapsulating polymerized microgels in the presence of empty droplets thereby selectively forming cell-encapsulating polymerized microgels. In theory, empty droplets will not undergo crosslinking due to the low solubility of the cross-linker in the empty droplets. In certain embodiments, cross-linking the emulsion results in a mixture of cell-encapsulating polymerized microgels and empty hydrogel particles, wherein the ratio of cell-encapsulating polymerized microgels to empty hydrogel particles is 60:40 to 99:1, 65:35 to 99:1, 70:30 to 99:1, 70:30 to 95:5, 70:30 to 90:10, 75:25 to 90:10, 80:20 to 90:10, 85:15 to 90:10, 70:30 to 85:15, 70:30 to 80:20, 75:25 to 85:15, 75:25 to 80:20, or 70:30 to 80:20.

As demonstrated in Example 6 below, the methods described herein can be adjusted to selectively encapsulate 1 cell, 2 cells, or more than 2 cells in each of the cell-encapsulating polymerized microgels in the emulsion by appropriate selection of the cell concentration and emulsion incubation time. Droplets containing a larger number of cells will achieve the necessary pH to induce alginate crosslinking in a shorter incubation time of the emulsion. Different incubation time of the emulsion (e.g., 1 h, 1,5 h, 2,5 h) results in different acid accumulation inside droplets. Thus, in certain embodiments, between 60-90%, 65-90%, 70-90%, 75-90%, 80-90%, 85-90%,60-85%, 65-85%, 70-85%, 75-85%, 80-85%, or 75-85% of the cell-encapsulating polymerized microgels in the emulsion contain a single cell. In certain embodiments, about 80% of the cell-encapsulating polymerized microgels in the emulsion contain a single cell.

In step (d), the emulsion is demulsified to obtain separated hydrogel particles and an aqueous phase. Demulsification can be carried out by such methods as adding the culture medium in the aqueous phase in the emulsion, adding a demulsifier, centrifuging or cryofreezing, etc. In certain embodiments of the method, demulsifiers that may be used are 1H,1H,2H,2H-perfluoro-1-octanol, any water phase that breaks the water-in-oil stable structure and the like.

Since the density of the hydrogel particles obtained after the alginate cross-linking is greater than that of the aqueous-phase droplets containing alginate, the hydrogel particles obtained after the cross-linking are able to be layered with the droplets in which no cross-linking reaction occurs. That is, the hydrogel particles obtained after cross-linking will settle to form the underlying sediment, while the droplets without cross-linking reaction will fuse to form the upper liquid after demulsification. Empty droplets (upper liquid) without cells can be removed to obtain the hydrogel particles that encapsulate cells, ensuring a higher cell encapsulation rate. After separation of the cell-encapsulating polymerized microgels is achieved, the hydrogel particles can be collected. The separation and collection of the hydrogel particles can adopt conventional methods in the art, e.g., centrifugation (density gradient centrifugation), sieving, surface adhesion, and the like.

The cell encapsulation method provided by the method described herein enables to obtain a deterministic cell encapsulation, and the obtained hydrogel particles encapsulates a definite number or type of cells.

It can be understood that the number of encapsulated cells in the droplet is positively correlated with the rate of accumulation of acidic material within the droplet, so that the droplet encapsulating two or more cells will cross-link more quickly than the droplet encapsulating a single cell, and a population of hydrogel particles encapsulating a target number of cells can be obtained by way of batch collection to finally achieve a deterministic cell encapsulation.

It is also possible to collect different kinds or types of cells in batches by using the difference in metabolic rates between different kinds or types of cells. Cancer cells, for example, produce large amounts of lactic acid even under aerobic conditions, and their acid accumulation efficiency is much higher than that of normal cells.

Thus, according to the cell encapsulation method of the present method, wherein each of the cell-encapsulating polymerized microgels obtained in step (d) comprises a single cell, two cells, or more than two cells:

In certain embodiments, step (d) comprises separating and collecting hydrogel particles encapsulating a single cell, hydrogel particles comprising two or more cells are collected first, and then the hydrogel particles encapsulating a single cell are separated and collected.

In certain embodiments, separating and collecting hydrogel particles encapsulating the first type of cells first, and then collecting and separating the hydrogel particles encapsulating the second type of cells, wherein the first type of cells can be cells with higher acid accumulation efficiency such as cancer cells, and the second type of cells can be cells with lower acid accumulation efficiency than the first type of cells, such as normal somatic cells and the like. Alternatively, the first type of cells may be living cells and the second type of cells may be dead cells.

In certain embodiments, in step (d), the hydrogel particles encapsulating individual cells are separated and collected to achieve deterministic single cell encapsulation. In certain embodiments, in step (d), hydrogel particles encapsulating two or more cells are separated and collected to achieve deterministic encapsulation of two or more cells.

In certain embodiments, in step (d) the hydrogel particles encapsulating living cells are separated and collected to achieve separation of dead and living cells.

In another aspect, the present disclosure also provides the cells or cell-encapsulating polymerized microgels prepared by the hydrogel-based cell encapsulation method as described above.

The cell-encapsulating polymerized microgels do not contain any labels and/or not affected by an external force field, so that the cells encapsulated therein are maximized to maintain their intrinsic behavior patterns and the intrinsic levels of various substances therein. The cells or cell-encapsulating polymerized microgels can be flexibly used for downstream operation and analysis, thereby further investigating such problems as the heterogeneities in proliferation, differentiation, metabolism and apoptosis at cell level.

In certain embodiments, the circularity of the cell-encapsulating polymerized microgels may be 0.5-1, or any value or range therebetween, e.g., 0.6, 0.7, 0.8, or 0.9.

In another aspect, the present disclosure also provides a hydrogel particle system prepared by the following method:
(a) preparing a cell suspension as an aqueous phase comprising cells, alginate and a culture medium;
(b) preparing an emulsion using the cell suspension and an oil phase through a droplet microfluidic device such that at least part of the droplets of the emulsion are encapsulated with cells; and
(c) incubating the emulsion, and then cross-linking alginate in the cell-encapsulating droplets in the presence of a cross-linker to form cell-encapsulating polymerized microgels.

In certain embodiments, the method further comprises (d) demulsification of the cell-encapsulating polymerized microgels.

In certain embodiments, the hydrogel particle system comprises more than 75%, more than 80%, or more than 90% of the cell-encapsulating polymerized microgels, for example, from 75% to 90% or from 75% to 85%; the hydrogel particle system comprises more than 70%, more than 75%, more than 80%, or more than 83% of hydrogel particles encapsulating individual cells, for example from 75% to 80% or from 75% to 90%.

In another aspect, the present disclosure also provides the use of the cell or cell-encapsulating polymerized microgel or hydrogel particle system as described above in the preparation of a formulation for use in cell research, such as cell life/death sorting, cell metabolism analysis, stem cell sorting, and liquid biopsy analysis. The cell research can be, for example, cell life/death sorting, cell metabolism analysis, stem cell sorting, single cell transcriptome sequencing, cell life/death analysis, and the like.

In certain embodiments, the cell-encapsulating polymerized microgels provided by the present disclosure may be used in relevant applications in the field of cell sorting. For example, the cell-encapsulating polymerized microgels may be obtained according to the above-described method by mixing cancer cells and somatic cells, and the difference in metabolic rate between cancer cells and somatic cells can be utilized to collect cancer cells first, thereby simulating the screening for the circulating tumor cells (CTC) in liquid biopsy for subsequent research to screen out the metabolic characteristics of the circulating tumor cells, providing a potential means for precision tumor treatment.

In another aspect, the present disclosure also provides a kit comprising:
alginate;
a culture medium;
an oil phase (e.g., which is selected from the group consisting of a fluorinated oil, a silicone oil, a rapeseed oil, a mineral oil, a commercial droplet oil (such as a droplet-forming oil sold by Biorad™), or any combination thereof);
a cross-linker, which is a divalent metal ion sulfate (e.g., calcium sulfate or barium sulfate); and
optional instructions for use.

In certain embodiments, in use, the cells are suspended in an aqueous solution comprising the alginate and culture medium to form a cell suspension. In certain embodiments, the divalent metal ion sulfate is in the form of a powder, a crystal, or any nanostructure (a nanotube, a nanoparticle, etc.).

In certain embodiments, the aqueous solution further comprises a water-soluble divalent metal salt, such as one or more of calcium chloride, calcium bromide, calcium iodide, calcium nitrate, calcium chlorate, calcium perchlorate, calcium bicarbonate, calcium dihydrogen phosphate, calcium acetate, calcium gluconate, calcium hydrogen phosphate, calcium lactate, calcium nitrate, barium chloride, barium sulfate, barium nitrate, barium carbonate, or barium cyanide.

In certain embodiments, the kit is used for performing the aforementioned hydrogel-based cell encapsulation method.

The aqueous solution, oil phase and cross-linker included in the kit may all use those as described above for the hydrogel-based cell encapsulation method described herein.

In certain embodiments, the divalent metal ion sulfate in the kit comprises calcium sulfate or barium sulfate. In certain embodiments, the calcium sulfate or barium sulfate is in the form of nanoparticles.

In certain embodiments of various aspects of the present disclosure, the culture medium is, for example, MEM (Minimum Essential Medium) or other common culture medium in the art, e.g., DMEM, RPMI1640, MEM, DMEM/F12, M199, IMDM, L15 Medium, etc., as well as improved mediums thereof. Those skilled in the art are able to select the specific composition and ratio of the corresponding cell culture medium according to the type of cells and/or for specific applications.

In the present disclosure, the term “optionally/optional” means that the features (e.g., components, steps, etc.) defined by the term may exist or not exist. For example, when referring to “optionally demulsifying, separating and collecting”, it means that the method may or may not include the steps of demulsifying, separating and collecting; when referring to “optional instructions for use”, it means that the instructions for use may or may not be included; and so on.

The present disclosure has at least the following advantages over conventional methods:
1. the hydrogel-based deterministic cell encapsulation method provided by the present disclosure enables a definite number (single cell or multiple cells, for example, 2, 3 or more cells) of cell encapsulation; it also enables to separate and collect hydrogel particles encapsulating different cells in batches based on the difference in metabolic rates of different cells for downstream analysis and research respectively; in addition, because the present method utilizes the metabolism of the cells themselves to achieve deterministic cell encapsulation and separation, the method described herein also enables to identify and separate living cells for subsequent research;
2. the hydrogel-based deterministic cell encapsulation method provided by the present disclosure enables a higher encapsulation rate;
3. the cells encapsulated in the cell-encapsulating polymerized microgels provided by the present disclosure are maximized to maintain their intrinsic behavior patterns and the intrinsic levels of various substances therein, avoiding errors in subsequent analysis of cells;
4. the hydrogel-based deterministic cell encapsulation method provided by the present disclosure has the characteristics of simple principle, friendly operation, and no need for the assistance of complicated instruments, not only has great significance for the research of the liquid droplet microfluidic device at cell level, but also can simplify the operation flow of cell research and has a wide application prospect in the fields of cell research and liquid biopsy.

Various embodiments of the present disclosure are described below. However, the embodiments are not limited to those described herein and may include, for example, derivations, variations, and/or modifications of the specific embodiments described herein.

### Example 1

### Droplet Microfluidic Device

The droplet microfluidic device used in this example might be a conventionally used droplet microfluidic device, which might be commercially available or may adopted a microfluidic chip fabricated according to the following steps.
1. Fabrication of the microfluidic chip template: the fabrication of the chip mold adopted general photolithographic technology. For example, a silicon wafer might be placed in a spin coater (Zhongke Micro-Control, KW-B-I), and about 5 ml of SU-8 2050 photoresist (MicroChem) was dripped at the center of the silicon wafer, and a suitable rotation speed was selected for spin coating to obtain a certain thickness. After standing for 5 mins, the silicon wafer was transferred onto a heating plate for pre-baking to cure it; the designed mask was bonded to the SU-8 photoresist, exposed under ultraviolet light to make it cross-linked, then transferred onto the heating plate for post-baking polymerization, and then slowly cooled to room temperature; finally, it was develop with a developer solution to remove the uncrosslinked photoresist to obtain the desired chip mold.
2. Fabrication of microfluidic chips: a certain amount of polydimethylsiloxane (PDMS) (Dow Corning Sylgard 184) (the mass ratio of PDMS precursor to cross-linker was 10:1) was prepared and stirred thoroughly to mix evenly. The chip template fabricated in step 1 was placed in a petri dish, poured a certain amount of PDMS, and placed in a vacuum dryer for degassing treatment; the petri dish, including the chip template and PDMS, was baked in an oven at 65° C. for at least 2 hours for curing; the PDMS patterned layer was peeled off the silicon wafer, and bonded with another PDMS substrate layer by plasma surface treatment after perforation at the fluid inlet and outlet. It was baked in an oven at 65° C. overnight for use.

### Preparation of Cell Suspension

A 10× MEM basal medium (M0275Sigma-Aldrich Minimum Eagle Medium, without L-glutamine and sodium bicarbonate, which was liquid and aseptically filtered, suitable for cell culture), 200 mM of L-glutamine, 10 g/L of glucose aqueous solution, and 100 mM of CaCl₂ aqueous solution were added sequentially and a suitable amount of water was added to obtain a solution with the final concentrations of 1×MEM, 2 mM of L-glutamine, 4.5 g/L of glucose, 6.54 mM of calcium chloride, mixed well. The pH value of the mixture was adjusted to around 7.3, and then 250 μL of 4 wt % sodium alginate aqueous solution was added and mixed evenly by gently blowing with a pipette gun to obtain 1 mL of sodium alginate-MEM aqueous solution with a final concentration of 1% for use.

A lung cancer A549 cell line was employed, a suitable basal medium was used, and a mixture of 10% FBS and 100 U/mL penicillin with 100 μg/mL streptomycin was added. When in use, the culture medium in the petri dish was poured off, 5 mL of PBS was added to rinse the cells, the waste liquid was aspirated, 1 mL of 0.25% trypsin/EDTA digestive solution was added, incubation was carried out at 37° C. for 1 min for digestion until the cells had just become round and floating, 5 mL of culture solution was added to stop the digestion, and was transferred to a 15 mL centrifuge tube, centrifuged at 1000 rpm for 3 min to aspirate the supernatant. The pre-prepared sodium alginate-MEM aqueous solution was added to resuspend the cells to make a cell suspension of lung cancer A549 cells with a cell concentration of 5×105 to 3×106 cells/ml for use.

In addition, white blood cells (WBCs) were employed to similarly make a cell suspension of the white blood cells with a cell concentration of about 5×10⁵ to 3×10⁶ cells/ml (e.g., about 5×10⁵ cells/ml, 1×10⁶ cells/ml or 3×10⁶ cells/ml) for use.

### Preparation of Calcium Sulfate Nanoparticles

In this example, calcium sulfate nanoparticles (particle size: 200-500 nm) were employed as the cross-linker. Calcium sulfate nanoparticles were prepared as follows: 3 mL of 5 wt % Triton X-100 in HFE-7500 was dissolved in 20 mL of cyclohexane and stirred for 30 min, after which 10 mL of 30 wt % H₂SO₄ aqueous solution was added to the mixture and stirred continuously for 3 h. Then, 0.3 g of CaCO₃ was added and stirring was continued overnight. The resulting suspension was centrifuged at 1500 rpm for 5 min, and immediately washed 3 times with absolute ethanol. The resulting solid product was dried in a vacuum oven at 65° C. for 4 hours and then collected to obtain calcium sulfate nanoparticles.

### Example 1—Deterministic Encapsulation of Cells

The oil phase of 1% (w/w) Triton X-100 in HFE-7500 (3M) and the cell suspension phase were injected into the microfluidic chip with a syringe at the flow rates of 6 μL/min and 2 μL/min, and a large number of uniform droplets were prepared and generated through the flow-focusing structure of the chip and collected into the well plate. After the collection was completed, they were transferred into the oven for the first incubation at 37° C. and 5% CO₂ for 2 hours, and then the mixture containing the calcium sulfate nanoparticles as prepared above and the oil phase was added, with the concentration of calcium sulfate nanoparticles being 0.1-1 g/ml, and the plate was placed on a shaker and shaken for the second incubation for 30 mins. Fresh medium was slowly added for demulsification and washing, cell-encapsulating polymerized microgels would be suspended and dispersed in the fresh medium, the cell-encapsulating polymerized microgels were separated and collected by centrifugation, and the collected particles are transferred onto clean well plates for subsequent culture experiments.

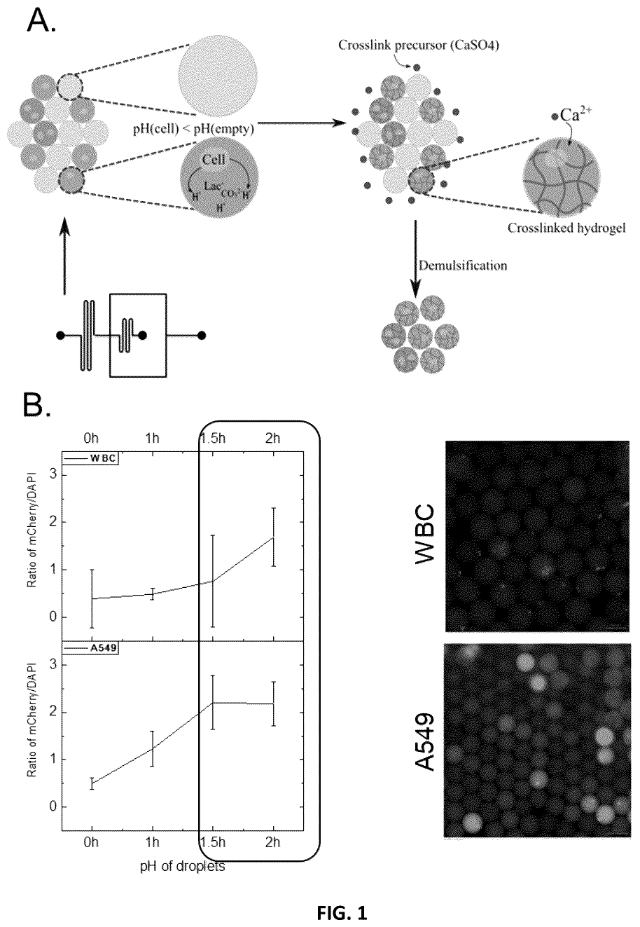

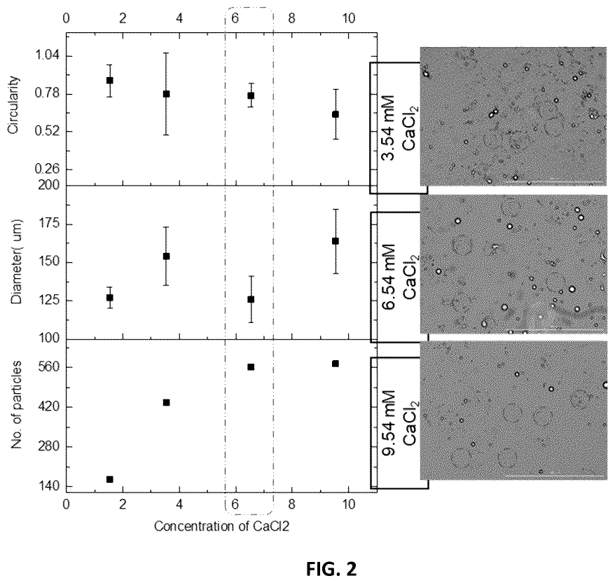

FIG. 1A shows a schematic flow chart of the deterministic cell encapsulation method described herein. As illustrated in FIG. 1, the cells were first dispersed in aqueous-phase droplets comprising sodium alginate and culture medium using a droplet microfluidic device. The droplets that encapsulated the cells were gradually acidified due to the accumulation of the cellular metabolites (e.g., lactate acid and carbon dioxide). The calcium sulfate nanoparticles may be added to the oil phase after the first incubation. The solubility of calcium sulfate increased with decreasing pH, and when the pH decreased to a certain level, sufficient calcium ions were dissolved into the alginate droplets, thereby triggering the droplets to polymerize into calcium alginate hydrogel particles. Subsequently, the empty droplets were removed and the cell-encapsulating polymerized microgels were separated by the demulsification process.

The lower panel B in FIG. 1 showed the use of media and fluorescent pH indicators to test pH changes in different cells due to acid accumulation. The two cell types, lung cancer A549 cell line and white blood cells, had different metabolism characteristics. Compared with somatic cells, in the presence of oxygen, compared with white blood cells (somatic cells), the lung cancer A549 cell line (cancer cells) preferred glycolysis instead of oxidative phosphorylation, leading to massive secretion of lactate instead of carbon dioxide.

In FIG. 1B, the left diagrams were the pH of the cell-encapsulating droplets obtained by multicolor fluorescence imaging as a function of the incubation time, and it could be seen that both types of cells reached the maximum pH change at 1.5-2 hours of incubation; the right pictures were the images of droplets encapsulating the white blood cells and lung cancer A549 cell line after the first incubation, respectively, and it could be seen that both types of cells showed accumulation of metabolic wastes, resulting in acidification of the droplets, showing droplets of different brightness, with the brighter droplets being those of lower pH.

### Example 2

In this example, the lung cancer A549 cell line was employed and the effect of the concentration of the water-soluble divalent metal salt CaCl₂ on the number, diameter, and circularity of particles was evaluated.

In this example, the crosslinking of the hydrogel was triggered by the decrease in pH caused by the accumulation of cell metabolites and the subsequent increase in the solubility of calcium sulfate. However, in cases where the increase in solubility was insufficient to fully crosslink the hydrogel, CaCl₂ might be added to the aqueous phase inside the droplet to obtain fully crosslinked hydrogel particles after the pH was lowered.

The steps similar to those in Example 1 were employed to obtain the cell-encapsulating polymerized microgels, except that the concentration of CaCl₂ in the cell suspension was varied from 1.5-9.5 mM during the preparation of the cell suspension. The results for the number, diameter, and circularity of the gel particles as a function of the CaCl₂ concentration are shown in FIG. 2.

The results showed that, as shown in FIG. 2, at relatively low concentrations, only a small amount of microgels could polymerize. With the increase of concentration, both the number and morphology of alginate particles improved. This indicated that the extra calcium ions provided by CaCl₂ might promote the formation and stabilization of hydrogel particles. However, if the concentration reached 10 mM, it might cause pre-gelation before the droplet was generated, and the circularity of microgels would become worse. This was because too much calcium ion would make the droplets in a semi-polymerized state before adding calcium sulfate, which would lead to uneven morphology in the droplet formation process. At the same time, at higher calcium chloride concentration, some empty droplets also polymerized into the microgels. Because the calcium ions inside were enough for hydrogel polymerization, it is no longer necessary to reduce pH to dissolve more calcium sulfate as a cross-linker. Therefore, it was important to select a suitable CaCl₂ concentration to obtain high encapsulation rate and circularity of gel particles. Suitable concentrations of CaCl₂ might be 2-8 mM, 3.5-7 mM, or 6.54 mM.

### Example 3

In this example, the lung cancer A549 cell line was employed and the effect of the first incubation time on the number, diameter, and circularity of gel particles was evaluated.

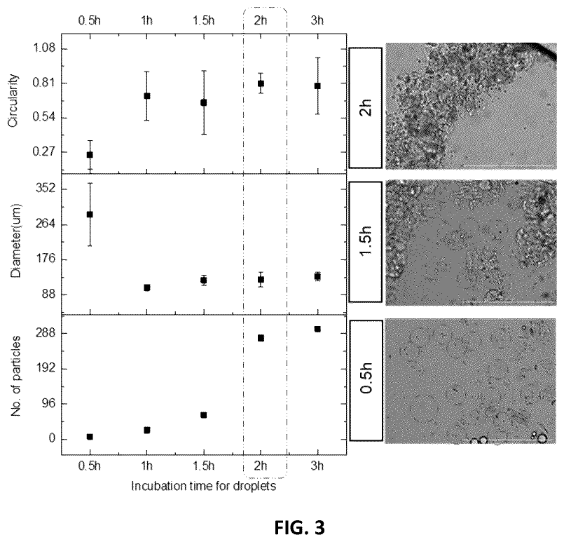

The steps similar to those in Example 1 were employed to obtain the cell-encapsulating polymerized microgels, except that the first incubation time was varied from 0.5-3 hours in the deterministic encapsulation step. The results obtained were shown in FIG. 3. As shown in FIG. 3, when the incubation time was only half an hour, it was difficult to find the regular microgels. This indicated that the acidification of the droplets was insufficient to initiate the polymerization of the hydrogel. The semi-crosslinked droplets fused during the demulsification process, resulting in the appearance of the irregular hydrogels. The incubation time was increased to obtain more acid accumulation, resulting in more microgels and more regular gel morphology. After incubation for 1.5 hours, the number of the particles increased sharply and better circularity could be obtained, and 2 hours was enough to acidify the droplets and gel the droplets into particles. After incubation for 3 hours, more hydrogel particles would be obtained than after incubation for 2 hours, but the gain was not obvious. Although more hydrogel particles might be obtained by longer incubation time, considering the cell viability would be affected if the cells were in the metabolic wastes for a long time, the suitable first incubation time might be 1.5-3 hours or 2-3 hours.

### Example 4

In this example, the lung cancer A549 cell line was employed and the effects of the cross-linker calcium sulfate and its different morphologies on the number, diameter and circularity of hydrogel particles were evaluated.

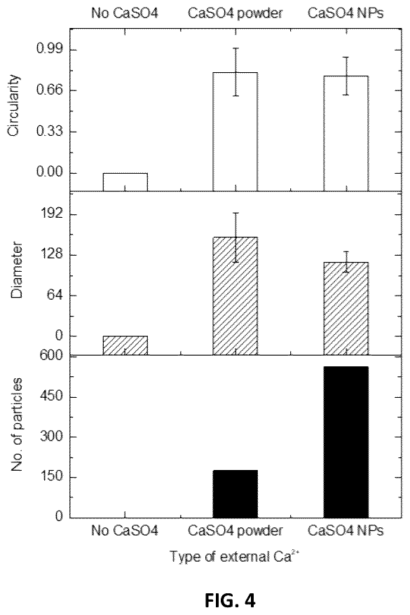

The steps similar to those in Example 1 were employed to obtain the cell-encapsulating polymerized microgels, except that the protocols without and with CaSO₄ powder added in the emulsion sample were employed respectively in the deterministic encapsulation step, and compared with the protocol with CaSO₄ nanoparticles (NP) added in the emulsion sample in Example 1. The results obtained were shown in FIG. 4.

As shown in FIG. 4, no gel particles were formed in the emulsion sample without CaSO₄ added, and a small amount of particles were formed in the emulsion sample with CaSO₄ powder added, while a large number of hydrogel particles were formed in the emulsion sample with CaSO₄ nanoparticles added, which was almost four times the number of gel particles formed in the emulsion sample with CaSO₄ powder added. Compared with the gel particles formed in the emulsion sample with CaSO₄ powder added, the particles in the emulsion sample with CaSO₄ nanoparticles added had lower and uniform diameters and equivalent circularity, which showed that the excellent dispersibility and smaller particle size of the CaSO₄ nanoparticles were beneficial to polymerization of the cell-encapsulating droplets into hydrogel.

### Example 5

In this example, the lung cancer A549 cell line was employed and the effect of the second incubation time on the number, diameter, and circularity of hydrogel particles was evaluated.

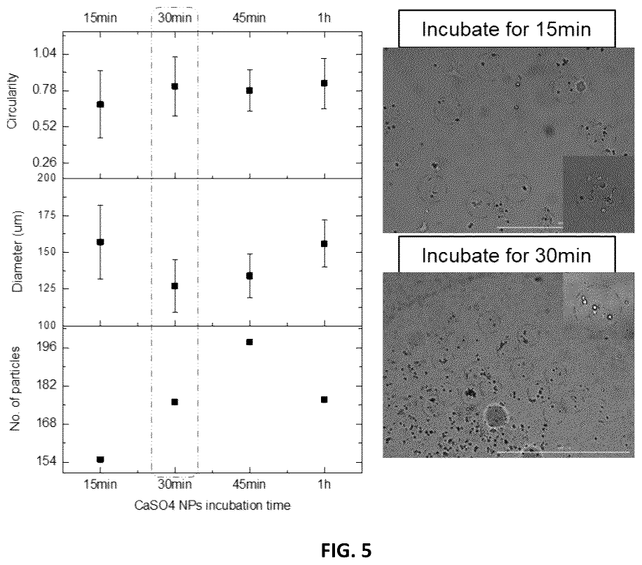

The steps similar to those in Example 1 were employed to obtain the cell-encapsulating polymerized microgels, except that the second incubation time was varied from 15 minutes to 1.5 hours in the deterministic encapsulation step. The results obtained were shown in FIG. 5. As shown in FIG. 5, appropriately prolonging the second incubation time with calcium sulfate was beneficial to the cross-linking of cell-encapsulating droplets, 30 minutes was enough to acidify the droplets and crosslink the droplets thereby forming cell-encapsulating polymerized microgels, and incubation for 45 minutes resulted in a significant increase in the number of cell-encapsulating polymerized microgels. However, after subsequent analysis, it was found that the longer incubation time made the empty droplets without cells also cross-linked, affecting the encapsulation rate. Considering the cell viability and the encapsulation rate, the second incubation time might be 20-40 minutes, 25-30 minutes or 30 minutes.

### Example 6

This example evaluated the effect of different cell concentrations on the encapsulation rate.

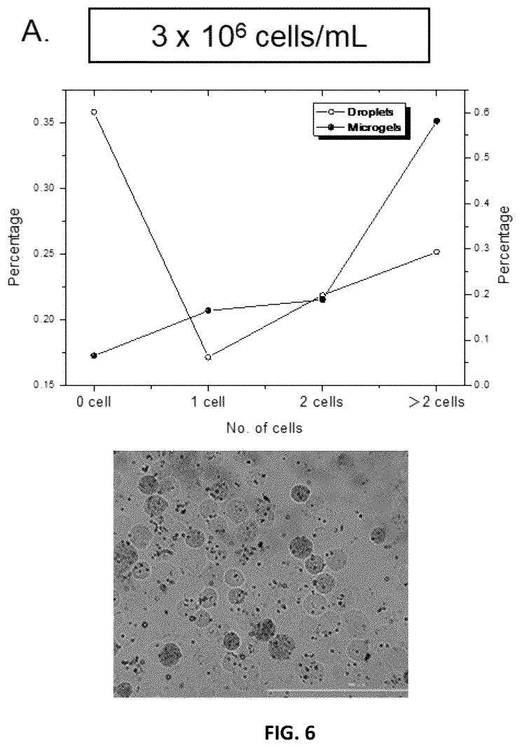

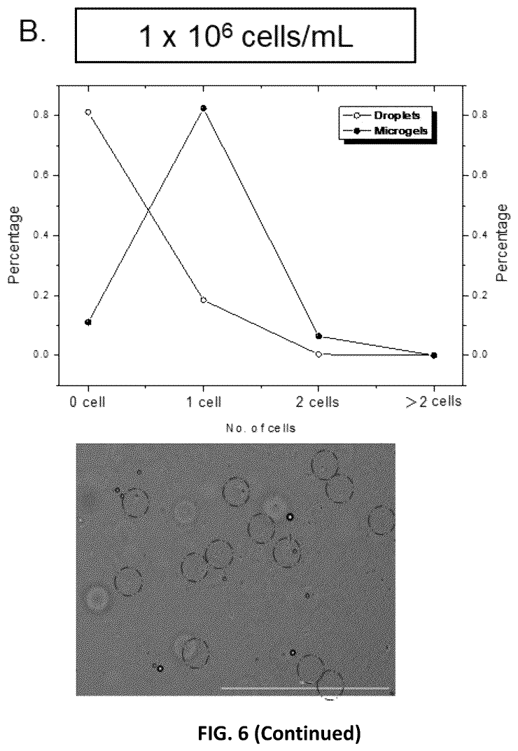

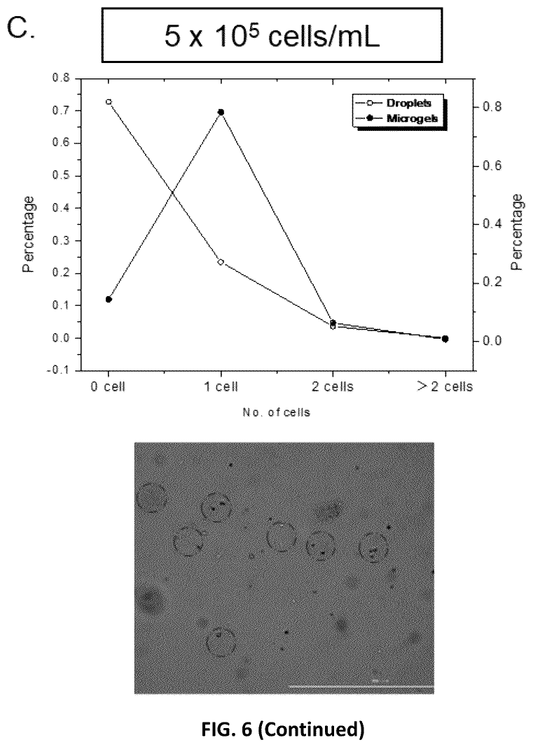

The steps similar to those in Example 1 were employed to obtain the cell-encapsulating polymerized microgels, except that cell concentrations of 3×10⁶ cells/ml, 1×10⁶ cells/ml and 5×10⁵ cells/ml were employed, respectively, in the preparation steps of the suspension of the lung cancer A549 cell line. The results for the encapsulation rate of the lung cancer A549 cell line in droplets and microgels at different concentrations were shown in FIGS. 6A, 6B and 6C. As shown in FIGS. 6A-6C, for the gel particles, a cell encapsulation rate of more than 90% could be achieved at the above-described concentrations, and a single-cell encapsulation rate of more than 80% was obtained at the cell concentrations of 1×10⁶ cells/ml and 5×10⁵ cells/ml, while the percentage of particles encapsulating single cells decreased at higher cell concentrations. In contrast, there was little or no encapsulation of cells in the vast majority of droplets.

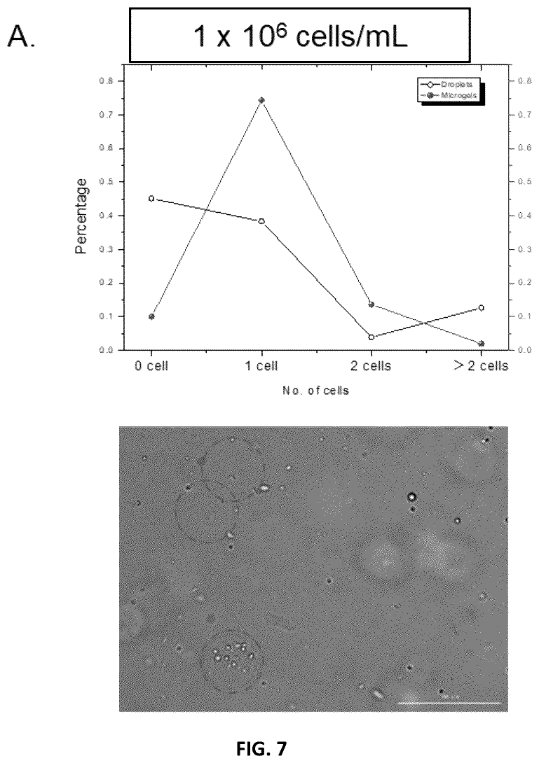

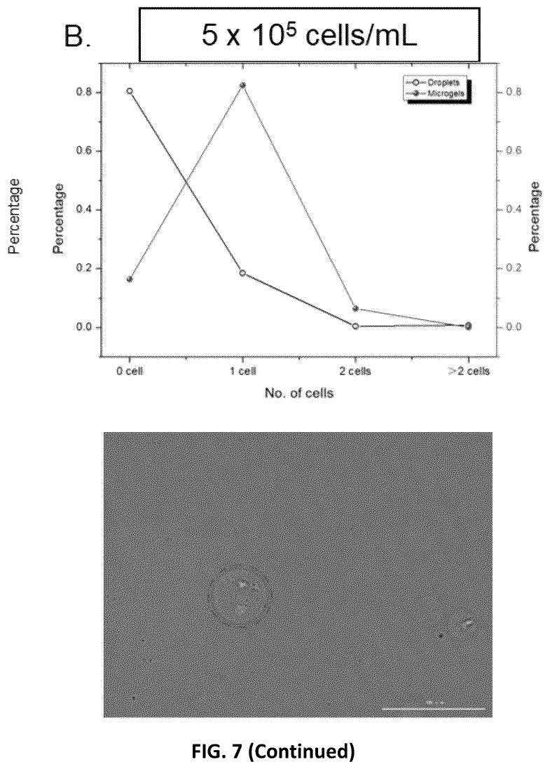

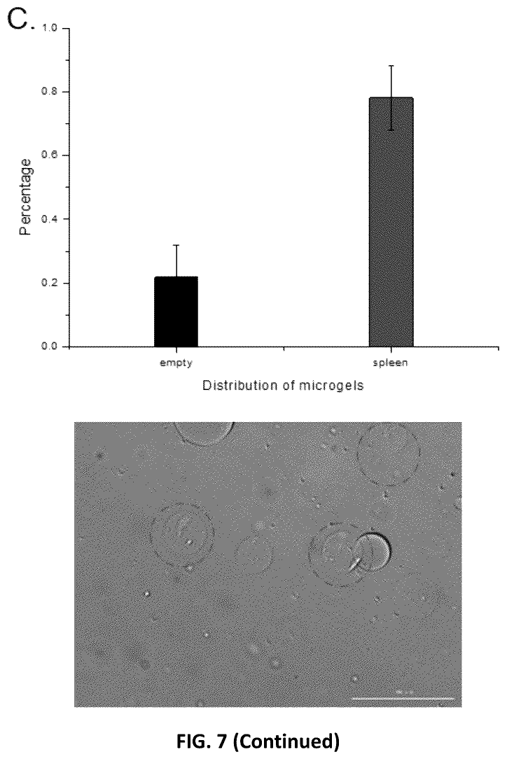

With cell concentrations of 1×10⁶ cells/ml and 5×10⁵ cells/ml, the steps similar to those in Example 1 were employed to obtain the hydrogel particles encapsulating white blood cells (WBCs). The encapsulation rate of WBCs in droplets and microgels at different concentrations was shown in FIGS. 7A and 7B. It could be seen that for the gel particles, the cell encapsulation rate of about 90% was obtained, wherein the single-cell encapsulation reached more than 75% and 85%, respectively. In contrast, the cell encapsulation rate in the droplets was much lower.

With a cell concentration of 5×10⁵ cells/ml, the steps similar to those in Example 1 were employed to obtain the hydrogel particles encapsulating spleen cells. The cell encapsulation rate of spleen cells was shown in FIG. 7C. It could be seen that the cell encapsulation rate of about 78% was achieved for the spleen cells, wherein the single-cell encapsulation rate was about 73.5%.

Therefore, regardless of the cell type, the method described herein increased the cell encapsulation rate to around 90%, and the single-cell encapsulation rate to around 80% or close to 80%.

### Example 7

In this example, the cell viability of the encapsulated cells after release was evaluated to demonstrate the potential of the method described herein to connect with other analysis platforms for downstream analysis and research.

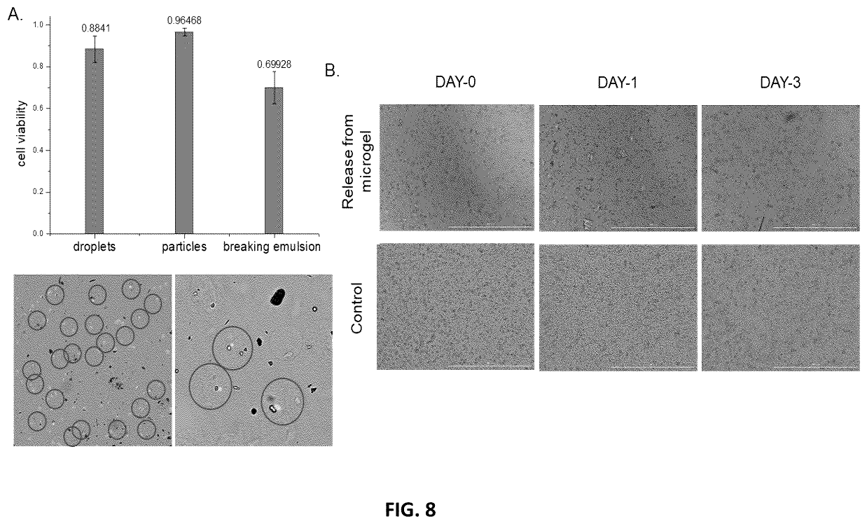

FIG. 8 showed the cell viability and cell proliferation experiments after release. As shown in A of FIG. 8, the cell viability in the droplet was around 88%, the cell viability in the gel particle can be as high as around 96%, and the cell viability after demulsification was around 70%. This indicated that the method described enabled to obtain cells after release with higher cell viability for downstream analysis and research. It also indicated that the method of the present disclosure enabled to screen and encapsulate living cells, and could be used for life/death screening applications of various types of cells (cancer cells, somatic cells, spleen cells, stem cells or germ cells, etc.).

As shown in B of FIG. 8, the hydrogel-encapsulated cells proliferated after release, and as shown in the images from day 0 to day 3, there was no significant difference in their proliferative ability compared with the control cells, which were cultured in normal condition.

In conclusion, the present disclosure provides an improved deterministic cell encapsulation method involving the use of simplified operational flow and experimental equipment setup. The present disclosure uses simple principles and methods to solve complex tasks that traditional methods require multi-steps and complex instruments to complete. Using the universal metabolism activities to recognize the droplets encapsulating cells not only avoids the possible side effects of labeling on cell function and complicated operation, but also removed dead cells at the same time to avoid interference with subsequent analysis. The separation is carried out by utilizing the difference of phase state between cell-encapsulating polymerized microgels and empty droplets so that the introduction of an external force field is avoided, and complex instruments and operation training are omitted. Meanwhile, compared with other methods that use the external force field to check on each droplet one by one, the batch sorting method is no longer limited by screening frequency. It not only has great theoretical significance for the research of droplet microfluidics at cell level but also simplifies the operation process and equipment requirement of cell-level research and has wide application prospects in cell research, tissue engineering, and regenerative medicine applications.

Although the present disclosure has been described in detail, modifications within the spirit and scope of the present disclosure will be apparent to those skilled in the art. It should be understood that aspects of the present disclosure and portions of various embodiments and various features recited above and/or in the appended claims may be combined or interchanged either in whole or in part. In the foregoing description of various embodiments, those embodiments that refer to another embodiment may be appropriately combined with other embodiments, as will be understood by those of ordinary skill in the art. In addition, those of ordinary skill in the art will understand that the above description is by way of example only, and is not intended to limit the present disclosure.

What is claimed is:

1. A hydrogel-based cell encapsulation method comprising:
(a) providing a cell suspension as an aqueous phase comprising cells, alginate and a culture medium;
(b) preparing an emulsion using the cell suspension and an oil phase through a droplet microfluidic device such that one or more of the cells are each encapsulated in at least a portion of the droplets thereby forming an emulsion comprising cell-encapsulating droplets and empty droplets;
(c) incubating the emulsion, and then cross-linking alginate in the cell-encapsulating droplets in the presence of a cross-linker to form cell-encapsulating polymerized microgels; and
(d) optionally, demulsifying, separating and collecting the cell-encapsulating polymerized microgels.

2. The cell encapsulation method of claim 1, wherein in step (a), the cell suspension further comprises a water-soluble divalent metal salt selected from the group consisting of calcium chloride, calcium bromide, calcium iodide, calcium nitrate, calcium chlorate, calcium perchlorate, calcium bicarbonate, calcium dihydrogen phosphate, calcium acetate, calcium gluconate, calcium hydrogen phosphate, calcium lactate, calcium nitrate, barium chloride, barium sulfate, barium nitrate, barium carbonate, barium cyanide, and a combination thereof.

3. The cell encapsulation method of claim 2, wherein the concentration of the water-soluble divalent metal salt in the cell suspension is 2-8 mM, 3.5-7 mM, or 6-7 mM.

4. The cell encapsulation method of claim 1, wherein the step (c) comprises: subjecting the emulsion to a first incubation for 0.5-24 hours or 1.5-3 hours; and subjecting the emulsion to a second incubation for 10-60 minutes or 15-35 minutes in the presence of a cross-linker, to crosslink the alginate in the cell-encapsulating droplets.

5. The cell encapsulation method of claim 4, wherein the cross-linker is added to the emulsion before the first incubation or to the emulsion after the first incubation and before the second incubation.

6. The cell encapsulation method of claim 4, wherein the cross-linker is added to the oil phase of the emulsion or to the aqueous phase of the emulsion.

7. The cell encapsulation method of claim 4, wherein in step (c), when the first incubation is completed, the pH of the cell-encapsulating droplet is less than or equal to 6.5 or has a pH between 6-6.5.

8. The cell encapsulation method of claim 1, wherein the cross-linker is calcium sulfate, barium sulfate, or a combination thereof.

9. The cell encapsulation method of claim 8, wherein the cross-linker is in the form of a powder, a crystal, or a nanoparticle.

10. The cell encapsulation method of claim 1, wherein each of the cell-encapsulating polymerized microgels obtained in step (d) comprise: a single cell, two cells, or more than two cells; and separating and collecting the cell-encapsulating polymerized microgels comprises collecting and separating the cell-encapsulating polymerized microgels comprising two cells and two or more cells first, and then collecting and separating the cell-encapsulating polymerized microgels encapsulating single cells.

11. The cell encapsulation method of claim 1, wherein the cells comprise human cells, mammalian cells, cancer cells, somatic cells, spleen cells, stem cells, or germ cells.

12. The cell encapsulation method of claim 1, wherein in step (a), the concentration of the cells in the cell suspension is 5×10⁵ to 3×10⁶ cells/ml.

13. The cell encapsulation method of claim 1, wherein in step (b), the oil phase is selected from the group consisting of a fluorinated oil, a silicone oil, a rapeseed oil, a mineral oil, a droplet oil, or any combination thereof.

14. The cell encapsulation method of claim 1, wherein the emulsion comprises cell-encapsulating polymerized microgels and empty hydrogel particles at a ratio between 75:25 to 90:10, respectively.

15. The cell encapsulation method of claim 1, wherein the 70-80% of the cell-encapsulating polymerized microgels comprise a single cell.

16. The cell encapsulation method of claim 1, wherein separating and collecting the cell-encapsulating polymerized microgels comprises separating the cell-encapsulating polymerized microgel based on their relative density.

17. The cell encapsulation method of claim 1, wherein separating and collecting the cell-encapsulating polymerized microgels comprises centrifugation, sieving, or surface adhesion.

18. A cell-encapsulating polymerized microgel prepared by the cell encapsulation method of

1.

19. A kit for conducting the cell encapsulation method of 1, the kit comprising:
alginate;
a culture medium;
an oil phase;
a cross-linker; and
optionally instructions for use.
